(19) United States
(12) Patent Application Publication
(10) Pub. No.: US 2016/0302647 A1
(43) Pub. Date: Oct. 20, 2016

(54) ENDOSCOPE IMAGING UNIT AND METHOD FOR ASSEMBLING ENDOSCOPE IMAGING UNIT

(71) Applicant: OLYMPUS CORPORATION, Tokyo (JP)

(72) Inventors: Tatsuya ORIHARA, Tokyo (JP); Minoru NAKAMURA, Tokyo (JP); Toru ISAKA, Tokyo (JP); Tsutomu SASAMOTO, Tokyo (JP)

(73) Assignee: OLYMPUS CORPORATION, Tokyo (JP)

(21) Appl. No.: 15/195,664

(22) Filed: Jun. 28, 2016

Related U.S. Application Data
(63) Continuation of application No. PCT/JP2015/063732, filed on May 13, 2015.

(30) Foreign Application Priority Data
May 16, 2014 (JP) 2014-102454

(51) Int. Cl. A61B 1/00; G02B 23/24

(52) U.S. Cl. CPC A61B 1/00096; A61B 1/0011; A61B 1/00163; G02B 23/2484

(57) ABSTRACT

This endoscope imaging device is to improve focus adjustment accuracy between an imaging element and an objective lens. This endoscope imaging device has an objective lens unit frame holding objective lenses, and an imaging element supporting frame fitted onto an outer side of the objective lens unit and holding an imaging element. The lateral cross-sectional shape of a fitted portion of an outer surface of the objective lens unit frame and the lateral cross-sectional shape of a fitted portion of an inner surface of the imaging element supporting frame are different, and in the fitted portions, a contact portion where said two frames come into contact, and a gap portion where said two frames do not come into contact are formed, and also the objective lens unit frame and the imaging element supporting frame are cemented by a photosetting glue filled in the gap portion.

### CROSS-REFERENCE TO RELATED APPLICATIONS

This application is a Continuation Application of International Application No. PCT/JP2015/063732 filed on May 13, 2015, which claims priority to Japanese Application No. 2014-102454 filed on May 16, 2014. The contents of International Application No. PCT/JP2015/063732 and Japanese application No. 2014-102454 are incorporated herein by reference in its entirety.

### TECHNICAL FIELD

The present invention relates to an endoscope imaging unit and a method for assembling an endoscope imaging unit which are to be applied to an endoscope.

### BACKGROUND ART

Conventionally, with regard to imaging devices, a variety of techniques which are related to assembling and focus adjustment (position adjustment) regarding the objective lens unit and the imaging element are disclosed. (See PTL 1, for example.

Especially, in a fixed-focus imaging device applied to a medical endoscope, the objective lens unit and the imaging element are respectively held by holding frames, and then cemented to be fixed by a thermosetting resin after the focus adjustment is performed precisely.

### CITATION LIST

### Patent Literature

{PTL 1} Japanese Unexamined Patent Application, Publication No. 2003-274231
{PTL 2} Japanese Unexamined Patent Application, Publication No. 2008-167334
{PTL 3} Japanese Unexamined Patent Application, Publication No. 2008-153881

### SUMMARY OF INVENTION

An aspect of the present invention is to provide endoscope imaging device comprising: an objective lens unit frame holding an objective lens; and an imaging element supporting frame fitted onto an outer side of the objective lens unit and holding an imaging element, wherein at least a lateral cross-sectional shape of a fitted portion, onto which the imaging element supporting frame is fitted, of an outer surface of the objective lens unit frame and at least a lateral cross-sectional shape of a fitted portion, into which the objective lens unit frame is fitted, of an inner surface of the imaging element supporting frame are different from each other, wherein when the objective lens unit frame is fitted with the imaging element supporting frame, in the fitted portions, a contact portion in which the outer surface of the objective lens unit frame and the inner surface of the imaging element supporting frame come into contact, and a gap portion in which the outer surface of the objective lens unit frame and the inner surface of the imaging element supporting frame do not come into contact are formed, wherein the objective lens unit frame and the imaging element supporting frame are cemented and then fixed by a photosetting glue filled in the gap portion.

### BRIEF DESCRIPTION OF DRAWINGS

FIG. 1 is a longitudinal sectional view showing an ent configuration an endoscope imaging device according to first embodiment of the present invention.

FIG. 2 is a perspective view showing an entire configuration of the endoscope imaging device according to the first embodiment of the present invention.

FIG. 3 is a front view showing an entire configuration of the endoscope imaging device according to the first embodiment of the present invention.

FIG. 4 is a perspective view showing an entire configuration of an endoscope imaging device according to a second embodiment of the present invention.

FIG. 5 is a front view showing an entire configuration of the endoscope imaging device according to the second embodiment of the present invention.

FIG. 6 is a perspective view showing an entire configuration of an endoscope imaging device according to a third embodiment of the present invention.

FIG. 7 is an exploded perspective view showing an entire configuration of the endoscope imaging device according to the third embodiment of the present invention.

FIG. 8 is a front view showing an entire configuration of the endoscope imaging device according to the third embodiment of the present invention.

### DESCRIPTION OF EMBODIMENTS

### First Embodiment

An endoscope imaging device according to a first embodiment of the present invention is described below with reference to the drawings.

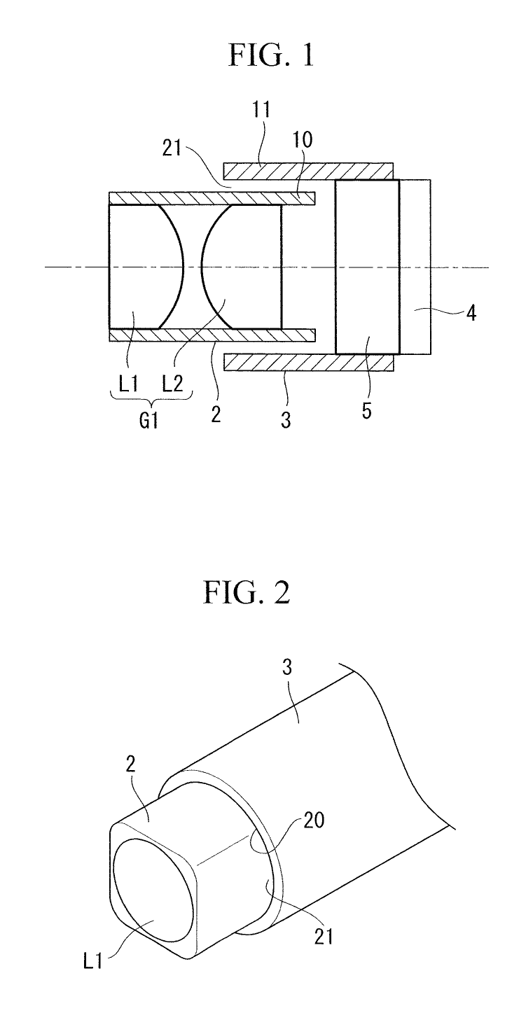

FIG. 1 is a longitudinal sectional view showing a schematic configuration of the endoscope imaging device according to the first embodiment. As shown in FIG. 1, the endoscope imaging device has an objective lens unit frame 2 for holding an objective lens, and an imaging element supporting frame 3 which holds an imaging element and into which the objective lens unit frame 2 is fitted.

As shown in FIGS. 1 and 2, the objective lens unit frame 2 is formed to a tubular shape which includes an outer surface which has a rectangular shape in the lateral cross-section and whose edges are chamfered to an R shape, and a penetrating hole which has a circular shape in the lateral cross-section, and the objective lens unit frame 2 has a group of lenses G1 which are located in the penetrating hole and which includes objective lenses L1, L2. The outer surface of one end of the objective lens unit frame 2 is a fitted portion 10 to be fitted with the imaging element supporting frame 3.

The imaging element supporting frame 3 is formed in a cylindrical shape, and one end of the inner hole thereof holds an imaging element 4 and a cover glass 5. The other end of the inner hole of the imaging element supporting frame 3 serves as a fitted portion 11 into which the objective lens unit frame 2 is fitted.

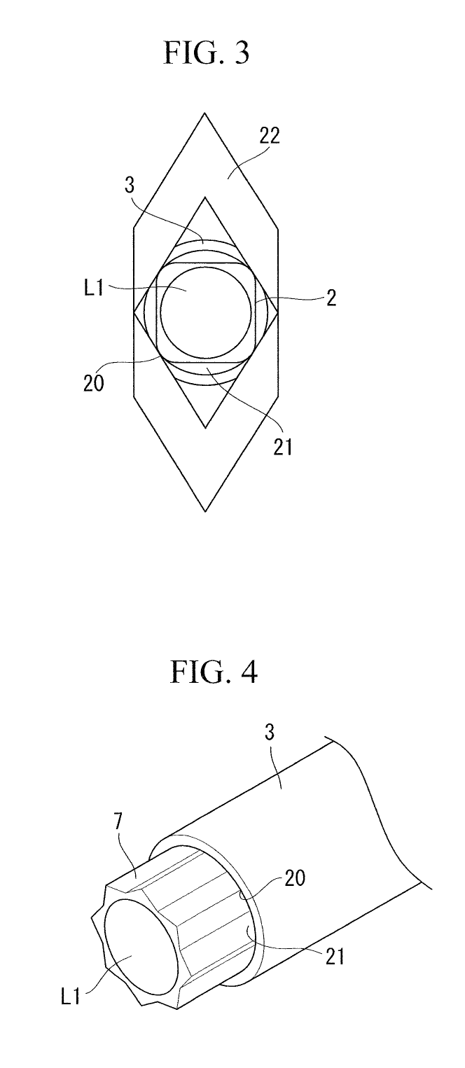

Thus, as shown in FIG. 3, the fitted portion 10, which is fitted into the imaging element supporting frame 3, of the outer surface of the objective lens unit frame is formed in a rectangular shape in the lateral cross-section with the edges chamfered, and the fitted portion 11, which is fitted with the objective lens unit frame 2, of the inner surface of the imaging element supporting frame 3 is formed in a circular shape in the lateral cross-section. In other words, the shapes of the fitted portions 10, 11 are different from each other in the lateral cross-section.

With this configuration, when the objective lens unit frame 2 is fitted into the imaging element supporting frame 3, in the fitted portions 10, 11, contact portions 20 in which the outer surface of the objective lens unit frame 2 and the inner surface of the imaging element holding portion 3 come into contact, and the gap portions 21 which is formed between the fitted portion 10 and the fitted portion 11 and in which the outer surface of the objective lens unit frame 2 and the inner surface of the imaging element supporting frame 3 do not come into contact are formed.

The gap portions 21 are filled with a photosetting glue, and thereby the objective lens unit frame 2 and the imaging element supporting frame 3 are cemented and then fixed.

The endoscope imaging device configured as described above is assembled as follows.

The fitted portion 10 of the objective lens unit frame 2 is fitted into the fitted portion 11 of the imaging element supporting frame 3 holding the imaging element. By this process, the chamfered edge portions of the fitted portion 10 of the objective lens unit frame 2 come into contact with the inner surface of the fitted portion 11 of the imaging element supporting frame 3, and thereby the contact portions 20 are formed. On the other hand, the portions between the edges of the fitted portions 10 of the objective lens unit frame 2 do not come into contact with the inner surface of the fitted portion 11 of the imaging element supporting frame 3, and thereby the gap portions 21 are formed between the fitted portion 10 and the fitted portion 11

The formed gap portions 21 are filled with the photosetting glue, and the focus adjustment between the objective lens unit frame 2 and the imaging element supporting frame 3 is performed. The focus adjustment is performed in a state in which the portions of the outer surface of the objective lens unit frame 2, which are located at the object surface side relative to the fitted portion 10 and at extended lines of the contact portions between the fitted portion 10 and the fitted portion 11, are held by a jig 22 or the like (FIG. 3). Then the photosetting glue is cured by irradiating light from the objective lens unit frame 2 side into the gap portions 21 along the optical axis of the objective lens.

As described above, with this embodiment, when the outer portion of the objective lens unit frame 2 is fitted into the imaging element supporting frame 3, where the surface of the fitted portion 10 and the surface of the fitted portion 11 have shapes in the cross section which are different from each other, in the fitted portions 10, 11, the outer surface of the objective lens unit frame 2 and the inner surface of the imaging element supporting frame 3 partially come into contact at the contact portions 20, and thereby light can be introduced from the optical axis of the objective lens. Therefore, it is possible to effectively irradiate the filled photosetting glue.

Especially, since the holding portions by the jig are portions on the extended lines of the contact portions 20 between the fitted portion 10 of the objective lens unit frame 2 and the fitted portion 11, the light irradiated into the gap portions 21 is not blocked by the jig, and therefore the gap portions 21 can be efficiently irradiated.

By this configuration, in a state in which the objective lens unit frame 2 and the imaging element supporting frame 3 are positioned and then fixed by the jig, when the light is irradiated into the gaps 21, it is possible to efficiently cure the photosetting glue in order that the objective lens unit frame 2 and the imaging element supporting frame 3 are cemented to be fixed. Therefore, it is possible to suppress positional deviation caused by thermal expansion of a jig or a component, and also to improve positioning accuracy between the objective lens and the imaging element by suppressing manufacturing error.

### Second Embodiment

An endoscope imaging device according to a second embodiment of the present invention is described below with reference to the drawings. In the following explanation, the configurations which are the same as those of the endoscope imaging device of the first embodiment described above are accompanied with the same reference signs, and the explanations thereof are omitted.

As shown in FIG. 4, the objective lens unit frame 7 is formed to a tubular shape which includes an outer surface which has a polygonal shape in the lateral cross-section, and a penetrating hole which has a circular shape in the lateral cross-section, the objective lens unit frame 7 has a group of lenses G1 which are located in the penetrating hole and which includes objective lenses L1, L2. The outer surface of one end of the objective lens unit frame 7 is the fitted portion 10 to be fitted with the imaging element supporting frame 3.

The imaging element supporting frame 3 is formed in a cylindrical shape, and one end of the inner hole thereof holds the imaging element 4 and the cover glass 5. The other end of the inner hole of the imaging element supporting frame serves as the fitted portion 11 into which the objective lens unit frame 7 is fitted.

As shown in FIG. 5, the fitted portion 10, which is fitted into the imaging element supporting frame 3, of the outer surface of the objective lens unit frame 7 is formed in a polygonal shape in the lateral cross-section, and the fitted portion, which is fitted with the objective lens unit frame 7, of the inner surface of the imaging element supporting frame is formed in a circular shape in the lateral cross-section. In other words, the shapes of the fitted portions 10, 11 are different from each other in the lateral cross-section.

When the objective lens unit frame 7 is fitted into the imaging element supporting frame 3, the maximum diameter portions of the cuter surface of the objective lens unit frame 7 become the contact portions 20 which come into contact with the inner surface of the imaging element supporting frame 3, and the portions between the adjacent contact portions 20 become the gap portions 21. The gap portions 21 are filled with the photosetting glue, and thereby the objective lens unit frame 7 and the imaging element supporting frame 3 are cemented and then fixed.

The endoscope imaging device configured as described above is assembled as follows.

The fitted portion 10 of the objective lens unit frame 7 is fitted into the fitted portion 11 of the imaging element supporting frame 3 holding the imaging element. By this process, the maximum diameter portions of the fitted portion 10 of the objective lens unit frame 7 come into contact with the inner surface of the fitted portion 11 of the imaging element supporting frame 3, and thereby the contact portions 20 are formed. On the other hand, the portions between the maximum diameter portions of the fitted portions 10 of the objective lens unit frame 7 do not come into contact with the inner surface of the fitted portion 11 of the imaging element supporting frame 3, and thereby the gap portions 21 are formed between the fitted portion 10 and the fitted portion 11.

The formed gap portions 21 are filled with the photosetting glue, and the focus adjustment between the objective lens unit frame 7 and the imaging element supporting frame 3 is performed. The focus adjustment is performed, in a state in which the portions of the outer surface of the objective lens unit frame 7, which are located at the object surface side relative to the fitted portion 10 and at extended lines of the contact portions between the fitted portion 10 and the fitted portion 11, are held by the jig 22 or the like (FIG. 5). Then the photosetting glue is cured by irradiating light from the objective lens unit frame 7 side into the gap portions 21 along the optical axis of the objective lens.

As described above, with this embodiment, since the surface of the fitted portion 10 and the surface of the fitted portion 11 have shapes in the cross section which are different from each other as described in the first embodiment, in the fitted portions 10, 11, the contact portions 20 where the outer surface of the objective lens unit frame 7 partially comes into contact with the inner surface of the imaging element supporting frame 3, and the gap portions 21 where the components are partially away from each other are formed. Further, the gap portions 21 are easily irradiated from the optical axis direction of the objective lens, thereby it is possible to effectively irradiate the filled photosetting glue. Therefore, it is possible to suppress positional deviation caused by thermal expansion of a jig or a component, and also to improve positioning accuracy between the objective lens and the imaging element by suppressing manufacturing error.

In the first embodiment and the second embodiment described above, although the imaging element supporting frame 3 is formed to a cylindrical shape, and the objective lens unit frames 2, 7 are shaped to a polygonal tubular shape, and then the shapes of the fitted portions 10, 20 in the lateral cross-section are different from each other, the shapes are not limited to such ones. For example, a configuration in which contact portions and gap portions are formed in the fitted portion when the objective lens unit frame is fitted to the imaging element supporting frame by forming the objective lens unit frame to a cylindrical shape, and forming the imaging element supporting frame to a polygonal tubular shape may be employed.

### Third Embodiment

An endoscope imaging device according to a third embodiment of the present invention is described below with reference to the drawings. In the following explanation, the configurations which are the same as those of the endoscope imaging device of the first embodiment described above are accompanied with the same reference signs, and the explanations thereof are omitted.

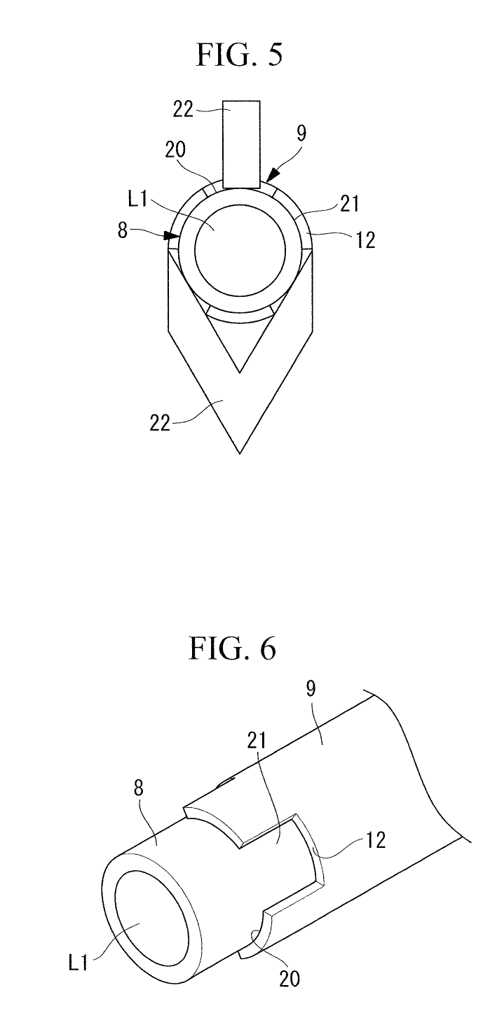

As shown in FIG. 6, the objective lens unit frame 8 is configured to a cylindrical shape, and the inner hole thereof holds a group of lenses G1 which includes the objective lenses L1, L2, The outer surface of one end of the objective lens unit frame 8 is the fitted portion 10 to be fitted with the imaging element supporting frame 9.

The imaging element supporting frame 9 is formed in a cylindrical shape, and one end of the inner hole thereof holds an imaging element 4 and a cover glass 5. The other end of the inner hole of the imaging element supporting frame 9 serves as the fitted portion 11 into which the objective lens unit frame 8 is fitted. The fitted portion 11 is provided with three cut portions with equal intervals so that each of the cut portions penetrates the imaging element supporting frame 9 in a direction crossing to the longitudinal axis direction.

As shown in FIG. 6, the fitted portion 10, which is fitted into the imaging element supporting frame, of the outer surface of the objective lens unit frame 8 is formed in a circular shape in the lateral cross-section with the edges chamfered, and the fitted portion, which is fitted with the objective lens unit frame 8, of the inner surface of the imaging element supporting frame 9 is formed in a three-arc shape which are not continuous in the lateral cross-section. In other words, the shapes of the fitted portions 10, 11 different from each other in the lateral cross-section.

When the objective lens unit frame 8 is fitted into the imaging element supporting frame 9, the outer surface of the fitted portion 10 of the objective lens unit frame 8 become the contact portions 20 which come into contact with the inner surface of the imaging element supporting frame 3. On the other hand, in the outer surface of the objective lens unit frame 8, the portions which exposes through the cut portions 12 of the fitted portions 11 of the imaging element supporting frame 9 serve as the gap portions 21. The gap portions 21 are filled with the photosetting glue, and thereby the objective lens unit frame 8 and the imaging element supporting frame 9 are cemented and then fixed.

The endoscope imaging device configured as described above is assembled as follows.

The fitted portion 10 of the objective lens unit frame 8 is fitted into the fitted portion 11 of the imaging element supporting frame 9 holding the imaging element. By this process, the outer surface of the fitted portion 10 of the objective lens unit frame 8 come into contact with the inner surface of the fitted portion 11 of the imaging element supporting frame 9, and thereby the contact portions 20 are formed. On the other hand, the portions of the fitted portion 10 of the objective lens unit frame 9, which does not come into contact with the inner surface of the fitted portion 11 of the imaging element holding portion 3 and which exposes through the cut portions 12, serve as the gap portions 21.

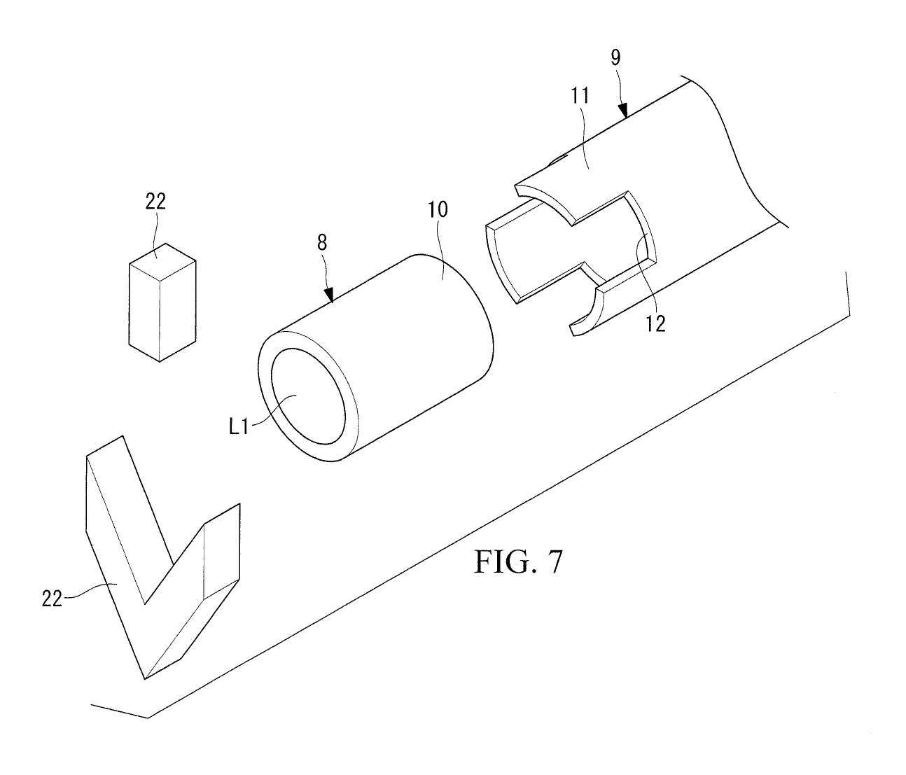

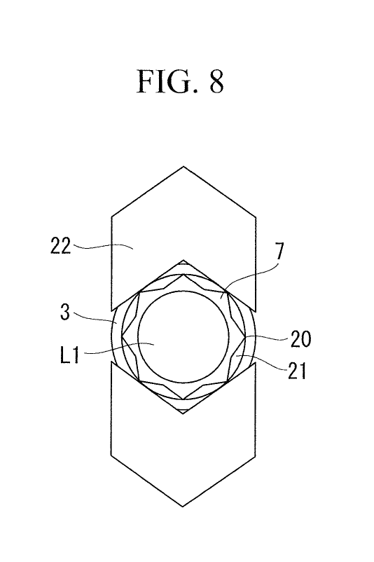

The gap portions 21 are filled with the photosetting glue, and the focus adjustment between the objective lens unit frame 8 and the imaging element supporting frame 9 is performed. The focus adjustment is performed in a state in which the portions of the outer surface of the objective lens unit frame 8, which are located at the object surface side relative to the fitted portion 10 and at extended lines of the contact portions between the fitted portion 10 and the fitted portion 11, are held by the jig 22 or the like. More specifically, as shown in FIGS. 7 and 8, the objective lens unit frame 8 and the imaging element supporting frame 9 are held by the jig 22 from a perpendicular direction of the optical axis of the objective lens so that the portions which is along the optical axis direction are exposed from the object surface side of the cut portions 12. Then the photosetting glue is cured by irradiating light from the objective lens unit frame 7 side into the gap portions 21 along the optical axis of the objective lens.

As described above, with this embodiment, since the surface of the fitted portion 10 and the surface of the fitted portion 11 have shapes in the cross section which are different from each other as described in the first embodiment, in the fitted portions 10, 11, the contact portions 20 where the outer surface of the objective lens unit frame 8 partially comes into contact with the inner surface of the imaging element supporting frame 9, and the gap portions 21 where the outer surface of the objective lens unit frame 8 expose are formed. Further, the gap portions 21 are easily irradiated from the optical axis direction of the objective lens, thereby it is possible to effectively irradiate the filled photosetting glue. Therefore, it is possible to suppress positional deviation caused by thermal expansion of a jig or a component, and also to improve positioning accuracy between the objective lens and the imaging element by suppressing manufacturing error.

The inventors have arrived at the following aspects of the invention.

An aspect of the present invention is to provide endoscope imaging device comprising: an objective lens unit frame holding an objective lens; and an imaging element supporting frame fitted onto an outer side of the objective lens unit and holding an imaging element, wherein at least a lateral cross-sectional shape of a fitted portion, onto which the imaging element supporting frame is fitted, of an outer surface of the objective lens unit frame and at least a lateral cross-sectional shape of a fitted portion, into which the objective lens unit frame is fitted, of an inner surface of the imaging element supporting frame are different from each other, wherein when the objective lens unit frame is fitted with the imaging element supporting frame, in the fitted portions, a contact portion in which the outer surface of the objective lens unit frame and the inner surface of the imaging element supporting frame come into contact, and a gap portion in which the outer surface of the objective lens unit frame and the inner surface of the imaging element supporting frame do not come into contact are formed, wherein the objective lens unit frame and the imaging element supporting frame are cemented and then fixed by a photosetting glue filled in the gap portion.

According to this aspect, when the imaging element supporting frame is fitted onto the objective lens unit frame while the cross-sectional shapes of the surfaces of the fitted portions of the two frames are different, in the fitted portion, the outer surface of the objective lens unit frame partially comes into contact with the inner surface of the imaging element supporting frame at the contact portion, and they are apart from each other at the gap portion. Also, light can be easily introduced in the gap portion from the optical axis direction of the objective lens, it is possible to effectively irradiate a photosetting glue filled in the gap portion. Therefore, in a state in which the objective lens unit frame and the imaging element supporting frame are positioned and then fixed by a jig, when the light is irradiated into the gap, it is possible to efficiently cure the photosetting glue in order that the objective lens unit frame and the imaging element supporting frame are cemented to be fixed.

As described above, when the photosetting glue is cured so that the objective lens unit frame and the imaging element supporting frame are cemented and the fixed, it only needs irradiating the gap, and it is not necessary to heat the whole structure while the objective lens unit frame and the imaging element supporting frame are fixed by the jig. Therefore, it is possible to suppress position aberration caused by thermal expansion of the jig or the parts, and thereby it is possible to improve positioning accuracy between the objective lens and the imaging element, that is, focus adjustment accuracy between the imaging element and the objective lens, reducing manufacturing errors.

In the above-described aspect, a cut portion which penetrates the imaging element supporting frame in a direction which crosses a longitudinal axis thereof may be provided in the fitted portion thereof.

With this configuration, the gap is easily formed in the fitted portion between the objective lens unit frame and the fitted portion of the imaging element supporting frame. Therefore, it is possible to apply the photosetting glue in the gap and irradiate the glue in order that the objective lens unit frame and the imaging element supporting frame are easily cemented to be fixed.

Another aspect of the present invention is to provide a method for assembling the endoscope imaging device according to claim 1 or 2, comprising: a step of fitting the objective lens unit frame holding the objective lens into an inner side of the imaging element supporting frame holding the imaging element; a step of filling a photosetting glue in the gap portion formed at the fitted portion between the objective lens unit frame and the imaging element supporting frame; a step of conducting focus adjustment between the objective lens unit frame and the imaging element supporting frame; and a step of irradiating the gap portion from an objective lens unit frame side along an optical axis of the objective lens so as to cure the photosetting glue.

As described above, since the photosetting glue is filled in the gap formed between the objective lens unit frame and the imaging element supporting frame, when the photosetting glue is cured, it is possible to irradiate the gap from the objective lens unit frame side along the optical axis of the objective lens. At the time of irradiation, when the objective lens unit frame and the imaging element supporting frame are fixed from a direction perpendicular to the optical axis of the objective lens, the irradiated light into the gap is not blocked by the jig and the like, therefore it is possible to efficiently irradiate the gap. Therefore, it is possible to suppress position aberration caused by thermal expansion of the jig or the parts, and thereby it is possible to improve focus adjustment accuracy between the imaging element and the objective lens, reducing manufacturing errors.

The aforementioned aspects can achieve an advantage of improving focus adjustment accuracy between an imaging element and an objective lens, reducing manufacturing errors.

### REFERENCE SIGNS LIST

2 objective lens unit frame
3 imaging element supporting frame
4 imaging element
5 cover glass
7 objective lens unit frame
8 objective lens unit frame
9 imaging element supporting frame
10 fitted portion
11 fitted portion
20 contact portion
21 gap portion
22 jig
G1 group of lenses
L1 objective lens
L2 objective lens

1. An endoscope imaging device comprising:
an objective lens unit frame holding an objective lens; and
an imaging element supporting frame fitted onto an outer side of the objective lens unit and holding an imaging element,
wherein at least a lateral cross-sectional shape of a fitted portion, onto which the imaging element supporting frame is fitted, of an outer surface of the objective lens unit frame and at least a lateral cross-sectional shape of a fitted portion, into which the objective lens unit frame is fitted, of an inner surface of the imaging element supporting frame are different from each other,
wherein when the objective lens unit frame is fitted with the imaging element supporting frame, in the fitted portions, a contact portion in which the outer surface of the objective lens unit frame and the inner surface of the imaging element supporting frame come into contact, and a gap portion in which the outer surface of the objective lens unit frame and the inner surface of the imaging element supporting frame do not come into contact are formed,
wherein the objective lens unit frame and the imaging element supporting frame are cemented and then fixed by a photosetting glue filled in the gap portion.

2. The endoscope imaging device according to claim 1,
wherein a cut portion which penetrates the imaging element supporting frame in a direction which crosses a longitudinal axis thereof is provided in the fitted portion thereof.

3. A method for assembling the endoscope imaging device according to claim 1, comprising:
a step of fitting the objective lens unit frame holding the objective lens into an inner side of the imaging element supporting frame holding the imaging element;
a step of filling a photosetting glue in the gap portion formed at the fitted portion between the objective lens unit frame and the imaging element supporting frame;
a step of conducting focus adjustment between the objective lens unit frame and the imaging element supporting frame; and
a step of irradiating the gap portion from an objective lens unit frame side along an optical axis of the objective lens so as to cure the photosetting glue.
